(19) United States
(12) Patent Application Publication
(10) Pub. No.: US 2011/0275698 A1
(43) Pub. Date: Nov. 10, 2011

(54) "TEST AND TREAT" STRATEGY FOR TREATING TRANSFORMING HPV INFECTION

(75) Inventors: Hanne Skomedal, Borgen (NO); Einar Morland, Klokkarstua (NO); Geir Morland, Klokkarstua (NO); Frank Karlsen, Klokkarstua (NO)

(73) Assignee: Norchip A/S, Klokkarstua (NO)

(21) Appl. No.: 12/776,643

(22) Filed: May 10, 2010

(51) Int. Cl. A61K 31/7105; A61P 35/00; A61P 31/20

(52) U.S. Cl. 514/44 A

(57) ABSTRACT

The invention is concerned with a “test and treat” method of screening and directly treating female subjects having transforming or abnormal human papillomavirus (HPV) infection.

### FIELD OF THE INVENTION

The invention is concerned with a “test and treat” method of screening and directly treating female subjects having transforming or abnormal human papillomavirus (HPV) infection.

### BACKGROUND TO THE INVENTION

Cervical carcinoma is one of the most common malignant diseases world wide and is one of the leading causes of morbidity and mortality among women. The current conception of cervical carcinoma is that it is a multistage disease, often developing over a period of 10-25 years. The clinical course of cervical carcinoma shows considerable variation; some patients with less favourable tumour characteristics have a relatively good outcome, while others suffer a fatal outcome of an initially limited disease.

Current guidelines from both the American College of Obstetricians and Gynecologists (ACOG) and the American Cancer Society recommend cervical screening, by conventional pap test or liquid-based cytology, annually or every 2-3 years for women >30 years of age with 3 negative cytology tests. Both agencies approve HPV DNA testing for reflex testing (triage) of women with ASC-US (atypical squamous cells of undetermined significance). If the HPV test is found to be positive, the subject is referred to colposcopy. Adjunct HPV typing is not currently recommended for subjects with LSIL (low-grade squamous intraepithelial lesion) or HSIL (high-grade squamous intraepithelial lesion).

Under the current guidelines for management of patients with cervical abnormalities, patients identified with ASC-US, LSIL and HSIL are typically referred for further evaluation by colposcopy, biopsy and histology. However, colposcopy-directed biopsy is highly subjective and the sensitivity and representivity of this technique is rather low (see abstract by Stoller, M. Accuracy and limitations of colposcopic performance. Eurogin 2010, February 17-20, Monte Carlo, Monaco, 2010, p 46).

One of the greatest challenges in developing cancer-screening guidelines is devising strategies that maximize screening benefits and minimize screening harms. The benefits of cancer screening—decreased cancer-related morbidity and mortality—are well known and widely promoted; the harms of cancer screening receive less attention and can take many forms: direct complications from screening and confirmatory tests; the expense, anxiety, and life disruptions incurred with new diagnoses with unclear clinical significance; and the prolonged surveillance endured by patients with positive morphological tests but no evidence of disease.

The gold standard of cervical pre-cancer diagnosis is morphological evaluation of biopsies from the cervix. However, the shortcomings of histological evaluation of colposcopy directed biopsies include, among others, low representivity, complex diagnostic interpretation, limited biopsy diagnosis coverage and poor reproducibility. Because biopsy collection is an invasive procedure, alternative methods would be desirable in order to improve primary screening for cervical cancer prevention. Histological examination has a very low sensitivity if only few biopsies from the cervix are collected. Alternative methods would be desirable in order to increase the representivity and the analytical and clinical sensitivity.

Human papillomavirus (HPV) infection is required, but not sufficient for the development of cervical cancer. The main discovery done by the Nobel Prize winner in Medicine in 2008, Harald zur Hausen, was the cause of cervical pre-cancer: The real driver of cervical dysplasia and cancer development is the production of E6 and E7 proteins following expression of E6/E7 mRNA from carcinogenic HPV types. The cause of cervical pre-cancer is the continuous production of E6 and E7 proteins following the presence of abnormal E6/E7 mRNA expression. The main cause of invasive cervical cancer is the continuous production of E6 and E7 proteins following the presence of abnormal E6/E7 mRNA expression. The presence of E6/E7 mRNA in the cervical mucosa or in the upper epithelium of the cervix is the due to loss of transcriptional regulation and integration of HPV. The presence of E6/E7 mRNA in the mucosa is technically the same as cervical pre-cancer, severe dysplasia or pre-cancer related intraepithelial neoplasia.

A number of studies have explored the potential role of HPV testing in cervical screening (see Cuzick et al. A systematic review of the role of human papillomavirus testing within a cervical screening programme. Health Technol Assess 3:14. 1999).

WO 99/29890 describes methods for the assessment of HPV infection based on the measurement and analysis of gene expression levels. In particular, WO 99/29890 describes methods which are based on measuring the levels of expression of two or more HPV genes (e.g. HPV E6, E7, L1 and E2) and then comparing the ratio of expression of combinations of these genes to provide an indication of the stage of HPV-based disease in a patient.

The present applicant has previously determined that it is possible to make a clinically useful assessment of HPV-associated disease based only on a simple positive/negative determination of expression of E6/E7 mRNA transcripts from carcinogenic/cancer-associated HPV types, with no requirement for accurate quantitative measurements of expression levels. This method is technically simple and, in a preferred embodiment, is amenable to automation in a mid-to-high throughput format. This method is described in detail in the applicant's published International application WO 03/57914.

The method described in WO 03/57914 is preferably carried out using the Pre-Tect HPV-Proofer™ kit, which is commercially available from Norchip AS, Klokkarstua, Norway. The HPV-Proofer assay provides four levels of information:

(1) Identification of mRNA from five specific different HPV-types (16, 18, 31, 33 and 45);
(2) Determination of the presence of oncogene HPV E6/E7 mRNA; and
(3) Determination the abnormal presence of HPV E6/E7 mRNA in the cervical mucosa or upper epithelium. In the normal viral life cycle the E6/E7 mRNA cannot be present in the cervical mucosa or upper epithelium due to the priority of the L1 and L2 related promoter (p670 or any other promoter downstream of the E6/E7 promoter p97). The production of E6/E7 mRNA in the upper epithelium is toxic to the viral particle production; and
(4) Determination of the presence of full length E6/E7 mRNA indicating dysregulation.

### SUMMARY OF THE INVENTION

The present inventors have evaluated the utility of a screen for E6/E7 mRNA transcripts from carcinogenic/cancer-associated HPV types as an indicator of the presence of cervical pre-cancer, also morphologically defined as high-grade dysplasia and cervical cancer (CIN2+). They have observed that the positive predictive value (PPV) of the E6/E7 mRNA test for histologically-defined CIN2+ cervical lesions is sufficiently high to justify direct treatment of those subjects who test positive for E6/E7 mRNA.

The present inventors have therefore developed a “test and treat” strategy for detecting and treating transforming HPV infections in human females, in which a treatment decision is made based on the outcome of a test for E6/E7 mRNA transcripts of at least one cancer-associated or carcinogenic HPV type. The “test and treat” approach can be used on women who exhibit normal or abnormal cervical cytology, particularly women with ASC-US, LSIL or HSIL cytology, and/or women who test positive for the presence of DNA from one or more cancer-associated/carcinogenic HPV types.

Therefore, in accordance with a first aspect of the invention there is provided a test and treat method of detecting and treating transforming infection with human papillomavirus (HPV) in a human female subject, the method consisting of:

testing a cervical sample from the subject for expression of E6/E7 mRNA transcripts from at least one cancer-associated (high risk) or carcinogenic HPV type;
making a treatment decision in a female human subject based on the outcome of the test for expression of E6/E7 mRNA transcripts of said at least one cancer-associated (high-risk) or carcinogenic HPV type, whereby a subject whose cervical sample tests positive for expression of E6/E7 mRNA transcripts from at least one cancer-associated (high risk) or carcinogenic HPV type is selected for direct treatment; and
treating said subject to reduce or eliminate transforming infection with human papillomavirus (HPV).

In accordance with a second aspect of the invention there is provided a test and treat method of screening (e.g. primary screening) a population of (e.g. normal) female human subjects, selecting and treating individuals identified as having transforming infection with human papillomavirus (HPV) based on the outcome of a test for expression of E6/E7 mRNA transcripts of human papillomavirus (HPV), the method consisting of:

testing cervical samples from subjects in the population for expression of E6/E7 mRNA transcripts from at least one cancer-associated (high risk) or carcinogenic HPV type;
making a treatment decision based on the outcome of the test for expression of E6/E7 mRNA transcripts from at least one cancer-associated or carcinogenic HPV type in cervical samples from said subjects, whereby a subject whose cervical sample tests positive for expression E6/E7 mRNA transcripts from at least one cancer-associated or carcinogenic HPV type is selected for direct treatment; and
treating said subject to reduce or eliminate transforming infection with human papillomavirus (HPV).

In accordance with a third aspect of the invention there is provided a test and treat method of triaging female subjects having a previous diagnosis of ASC-US, low-grade cervical lesions (LSIL) or high-grade cervical lesions (HSIL) by cytology, selecting and treating individuals identified as having transforming infection with human papillomavirus (HPV) based on the outcome of a test for expression of E6/E7 mRNA transcripts of human papillomavirus (HPV), the method consisting of:

testing a cervical sample from a subject having a previous diagnosis of ASC-US, low-grade cervical lesions (LSIL) or high-grade cervical lesions (HSIL) by cytology for expression of E6/E7 mRNA transcripts from at least one cancer-associated (high risk) or carcinogenic HPV type;
making a treatment decision based on the outcome of the test for expression of E6/E7 mRNA transcripts from at least one cancer-associated (high risk) or carcinogenic HPV type in said cervical sample, whereby a subject whose cervical sample test positive for expression of E6/E7 mRNA transcripts from at least one cancer-associated or carcinogenic HPV type is selected for direct treatment; and
treating said subject to reduce or eliminate transforming infection with human papillomavirus (HPV).

In accordance with a fourth aspect of the invention there is provided a test and treat method of triaging female subjects who previously tested positive for the presence of DNA from at least one cancer-associated (high risk) or carcinogenic HPV type, selecting and treating individuals identified as having transforming infection with human papillomavirus (HPV) based on the outcome of a test for expression of E6/E7 mRNA transcripts of human papillomavirus (HPV), the method consisting of:

testing a cervical sample from a subject who previously tested positive for the presence of DNA from at least one cancer-associated (high risk) or carcinogenic HPV type for expression of E6/E7 mRNA transcripts from at least one cancer-associated (high risk) or carcinogenic HPV type;
making a treatment decision based on the outcome of the test for expression of E6/E7 mRNA transcripts from at least one cancer-associated or carcinogenic HPV type in said cervical sample, whereby a subject whose cervical sample tests positive for expression E6/E7 mRNA transcripts from at least one cancer-associated or carcinogenic HPV type is selected for direct treatment; and
treating said subject to reduce or eliminate transforming infection with human papillomavirus (HPV).

### DEFINITIONS

“Transforming infection” with human papillomavirus is defined as an HPV infection characterised by persistent expression of E6/E7 mRNA transcripts, particularly E6/E7 mRNA transcripts encoding a full length E6 protein. Such infection is “abnormal” in that it represents a variation on the “normal” cycle of transient HPV infection.

“Cancer-associated” HPV type means a type of human papillomavirus known to be associated with cancer of the cervix. “Carcinogenic” HPV type means a type of human papillomavirus which has been classified as carcinogenic (in relation to cancer of the cervix) by the International Agency for Research on Cancer (IARC) monograph. “High risk” HPV type means a type of human papillomavirus which is highly likely to give rise to cancer of the cervix.

“E6/E7 mRNA” means all naturally occurring mRNA transcripts which contain all or part of the E6 open reading frame, including naturally occurring splice variants, and transcripts which contain all or part of the E7 open reading frame.

The term “full length E6/E7 mRNA transcripts” means any E6/E7 transcript which encodes a full length E6 protein. This definition excludes any of the naturally occurring splice variants, but encompasses bicistronic transcripts that encode functional full length E6 and E7 proteins.

A “test and treat” method is a strategy for management of transforming infection with cancer-associated (high risk) or carcinogenic human papillomavirus which combines molecular diagnostics and therapy. A molecular diagnostic test, based on screening for expression of E6/E7 mRNA transcripts of defined cancer-associated (high risk) or carcinogenic HPV types, is used to identify human female subjects for immediate treatment aimed at reducing/eliminating the HPV infection. Subjects testing positive for E6/E7 mRNA are directly/immediately treated with recourse to further testing or monitoring.

### DETAILED DESCRIPTION OF THE INVENTION

The present inventors have evaluated the performance of an assay for expression of E6/E7 mRNA transcripts of one or more cancer-associated (high risk) or carcinogenic HPV types in the context of the cervical screening program by making comparisons results of conventional cytology and histological examination of cervical abnormalities, as well as HPV DNA testing.

As a consequence, the present inventors have determined that the E6/E7 mRNA-based assay exhibits a high positive predictive value (PPV) for histologically-defined CIN2+ cervical lesions. In addition, and due to the combination of very high analytical sensitivity and an estimated high sensitivity for persistent CIN2+, they have observed that the rate of false negative results for the E6/E7 mRNA-based assay is extremely low. The PPV of the E6/E7 mRNA-based assay is equivalent to some of the patient groups selected to receive treatment under the current cervical screening guidelines. In fact, the discovery of such a high PPV in studies biased by the weakness of colposcopy and the morphological methods show that it will be difficult to claim any false positives based on the E6/E7 mRNA-based assay. The present inventors therefore surmise that the PPV calculated against histologically-defined CIN2+ cervical lesions is alone sufficiently high to justify direct treatment, by conisation or equivalent treatment, of those subjects who test positive in an assay for E6/E7 mRNA of at least one cancer-associated (high risk) or carcinogenic HPV type.

The highest positive predictive value was observed when the potential largest area of the cervical epithelium were covered by CIN2 or CIN3 like cells. The potential largest area may be discovered when abnormal pap test or liquid-based cytology test is positive as e.g. ASC-US, LSIL or HSIL. The highest PPV for the E6/E7 mRNA assay was by this reason discovered within cytological HSIL positive cases. The HSIL cases would represent the largest area of the epithelium covered by CIN2 or CIN 3 like cells making the pre-cancer cells more likely to be detected.

It means that any presence of E6/E7 mRNA within any epithelium cell in the cervix discovered by any collection device would represent a CIN2 or CIN3 like cell or a pre-cancer lesion. The probability of the existence of an abnormal pre-cancer cell or lesion in a population diagnosed to be cytological normal but having the presence of E6/E7 mRNA is the same as the existence of an abnormal pre-cancer cell or lesion in a population positive by abnormal cytology. The difference is only the number of abnormal pre-cancer cells present. It may often happen that pathologist may diagnose a case to be CIN2, ASCUS-H or HSIL just by detection of very few abnormal cells.

Therefore, a first aspect of the invention relates to a “test and treat” method of detecting and treating transforming infection with human papillomavirus (HPV) in a human female subject, the method consisting of:

testing a cervical sample from the subject for expression of E6/E7 mRNA transcripts from at least one cancer-associated (high risk) or carcinogenic HPV type;
making a treatment decision in a female human subject based on the outcome of the test for expression of E6/E7 mRNA transcripts of said at least one cancer-associated (high-risk) or carcinogenic HPV type, whereby a subject whose cervical sample tests positive for expression of E6/E7 mRNA transcripts from at least one cancer-associated (high risk) or carcinogenic HPV type is selected for direct treatment; and
treating said subject to reduce or eliminate transforming infection with human papillomavirus (HPV).

This method involves testing a cervical sample from a human female subject for expression of E6/E7 mRNA transcripts of said at least one cancer-associated (high-risk) or carcinogenic HPV type and then making a treatment decision based on the outcome of this test. Subjects testing positive for expression of E6/E7 mRNA transcripts from at least one cancer-associated (high-risk) or carcinogenic HPV type are immediately identified as requiring treatment. Such subjects are then directly treated, meaning that they are subjected to treatment without recourse to any further monitoring, screening or diagnostic testing related to cervical abnormalities and/or HPV infection. In this context, and indeed all embodiments of the invention, the term “treatment” refers to any treatment which reduces or eliminates transforming infection with human papillomavirus, including treatments aimed at eliminating or reducing the numbers of abnormal, e.g. pre-cancerous, cervical cells. Suitable treatments include conisation or cone biopsy and equivalent treatments, also treatments with pharmaceutical agents, drugs, small-molecules or RNA (miRNA, siRNA treatment).

In certain embodiments, the “test and treat” method of the invention can be applied to human female subjects having no previous history of cervical abnormalities. Such subjects could include individuals who have never been enrolled in the cervical screening program and also individuals who have been screened using cytological methods, e.g. pap test or liquid-based cytology, but have never shown any cytological abnormality.

A particular application of the “test and treat” method of the invention may be in routine screening (e.g. primary screening) of a population of human female subjects. Accordingly, a particular embodiment of the invention relates to a test and treat method of screening a population of female human subjects, selecting and treating individuals identified as having transforming infection with human papillomavirus (HPV) based on the outcome of a test for expression of E6/E7 mRNA transcripts of human papillomavirus (HPV), the method consisting of:

testing cervical samples from subjects in the population for expression of E6/E7 mRNA transcripts from at least one cancer-associated (high risk) or carcinogenic HPV type; making a treatment decision based on the outcome of the test for expression of E6/E7 mRNA transcripts from at least one cancer-associated (high risk) or carcinogenic HPV type in cervical samples from said subjects, whereby a subject whose cervical sample tests positive for expression E6/E7 mRNA transcripts from at least one cancer-associated or carcinogenic HPV type is selected for direct treatment; and
treating said subject to reduce or eliminate transforming infection with human papillomavirus (HPV).

In this screening embodiment, subjects may be selected for treatment solely on the basis of the outcome of the test for E6/E7 mRNA expression, individuals whose cervical sample tests positive for expression E6/E7 mRNA transcripts from at least one cancer-associated (high risk) or carcinogenic HPV type is selected for direct treatment without recourse to the results of cytology tests (past or concurrent). In this context, “treatment” again refers to any treatment which reduces or eliminates transforming infection with human papillomavirus, including treatments aimed at eliminating or reducing the numbers of abnormal, e.g. pre-cancerous, cervical cells. Suitable treatments include conisation or cone biopsy and equivalent treatments, also treatments with pharmaceutical agents, drugs, small-molecules or RNA (miRNA, siRNA treatment).

A “test and treat” methodology for primary screening, and subsequent direct treatment of individuals identified as in need of treatment, is possible if the assay for E6/E7 mRNA expression, when applied to a test population with no history of cytological abnormality, gives a sufficiently high PPV for high grade cervical lesions which would progress to cervical carcinoma if left untreated, e.g. histologically confirmed CIN2+ or CIN3+. The present inventors have observed that this is indeed the case for an assay based on detection of E6/E7 mRNA expression, meaning that the assay for E6/E7 mRNA expression has potential for use in primary screening, i.e. without the need for cytology or colposcopy.

In further embodiments, the “test and treat” method of the invention can be applied to human female subjects who already manifest cervical abnormalities based on cytology, e.g. pap test or liquid-based cytology. In particular, the method could be applied to human female subjects exhibiting cytological abnormalities classified as ASC-US (atypical squamous cells of undetermined significance), LSIL (low-grade squamous intraepithelial lesion) or HSIL (high-grade squamous intraepithelial lesion).

Therefore, a particular embodiment of the invention provides a test and treat method of triaging female subjects having a previous diagnosis of ASC-US, low-grade cervical lesions (LSIL) or high-grade cervical lesions (HSIL) by cytology, selecting and treating individuals identified as having transforming infection with human papillomavirus (HPV) based on the outcome of a test for expression of E6/E7 mRNA transcripts of human papillomavirus (HPV), the method consisting of:

testing a cervical sample from a subject having a previous diagnosis of ASC-US, low-grade cervical lesions (LSIL) or high-grade cervical lesions (HSIL) by cytology for expression of E6/E7 mRNA transcripts from at least one cancer-associated (high risk) or carcinogenic HPV type;
making a treatment decision based on the outcome of the test for expression of E6/E7 mRNA transcripts from at least one cancer-associated (high risk) or carcinogenic HPV type in said cervical sample, whereby a subject whose cervical sample test positive for expression of E6/E7 mRNA transcripts from at least one cancer-associated (high risk) or carcinogenic HPV type is selected for direct treatment; and
treating said subject to reduce or eliminate transforming infection with human papillomavirus (HPV).

In this triage embodiment, subjects identified as having cervical abnormalities based on conventional cytology may be selected for treatment solely on the basis of the outcome of a test for E6/E7 mRNA expression, individuals whose cervical sample tests positive for expression E6/E7 mRNA transcripts from at least one cancer-associated (high risk) or carcinogenic HPV type is selected for direct treatment without the need for further investigation. In this context, “treatment” again refers to any treatment which reduces or eliminates transforming infection with human papillomavirus, including treatments aimed at eliminating or reducing the numbers of abnormal, e.g. pre-cancerous, cervical cells. Suitable treatments include conisation or cone biopsy and equivalent treatments, also treatments with pharmaceutical agents, drugs, small-molecules or RNA (miRNA, siRNA treatment).

The “test and treat” triage method may avoid the need to perform colposcopy-directed biopsy and histology as a matter of routine on all patients who exhibit abnormalities based on conventional cytology. This would be a significant advantage as colposcopy-directed biopsy is expensive, subjective and lacking in sensitivity and representivity and also invasive/uncomfortable for the patient. The majority of mild abnormalities identified by conventional cytology are unrelated to cervical cancer and will go away without treatment, but some may lead to a precancerous condition or cancer. The “test and treat” triage method of the invention can avoid the need to investigate all patients exhibiting mild cytological abnormalities by colposcopy, and instead identify a subset of those individuals with mild cytological abnormalities in which the abnormality is high likely to be related to cervical pre-cancer. In these cases, the present inventors have observed that the PPV of the E6/E7 mRNA for pre-cancerous lesions is sufficiently high to warrant direct treatment of mRNA-positive individuals, without recourse to expensive, and potentially unreliable, investigation by colposcopy.

The “test and treat” method may also avoid the need for colposcopic examination. Colposcopic examination in a screening population may be positive on a high number of cases, causing a very high number of false positive cases. Many gynaecologists world-wide are using colposcopy as a triage before cytology or HPV DNA testing. These high number of positive colposcopic examination is making many women unnecessary anxious

A “test and treat” methodology for triage of subjects with mild cervical abnormalities, e.g. ASC-US or LSIL, and subsequent direct treatment of individuals identified as in need of treatment, is possible if the assay for E6/E7 mRNA expression, when applied to a test population of individuals with mild cervical abnormalities (ASC-US and LSIL), gives a sufficiently high PPV for high grade cervical lesions which would progress to cervical carcinoma if left untreated, e.g. histologically confirmed CIN2+ or CIN3+. The present inventors have observed that this is indeed the case for an assay based on detection of E6/E7 mRNA expression, meaning that the assay for E6/E7 mRNA expression has potential for use in triage of subjects presenting with ASC-US or LSIL, without the need for further investigation by colposcopy and/or management of the patient by ongoing cytological examination.

The test and treat triage method is particularly advantageous if the initial cervical cytology is carried out using liquid-based cytology rather than by pap smear, since the same liquid-based cytology sample can be used to test for E6/E7 mRNA expression.

In a still further embodiment, the “test and treat” method of the invention could be applied to human female subjects who have previously tested positive for the presence of HPV DNA from one or more cancer-associated (high-risk) or carcinogenic HPV types, with or without cervical abnormalities assessed by cytology.

An additional advantage of the “test and treat” strategy is that it minimises the risk of “losing” women in the follow-up procedure following a positive cytology result. Clinical studies have shown that a significant number of women with positive cytology are “lost” in follow-up. It is therefore very important to reduce the number of recalls by offering treatment as soon as possible to those women at highest risk, because of active expression of E6/E7 mRNA from cancer-associated (high risk) HPV types.

In the following passages particular features common to all embodiments/aspects of the methods of the invention are defined/described in further detail. Unless otherwise stated, features identified as being particularly preferred apply to all embodiments/aspects of the invention.

In all embodiments of the method of the invention, the step of testing cervical samples for expression of E6/E7 mRNA transcripts from at least one cancer-associated (high-risk) or carcinogenic HPV type may be carried out using assay methodology already known in the art. Suitable techniques are described in WO 03/057914 and WO 2005/083129, both of which are incorporated herein entirely by reference.

The cervical sample must comprise at least one cervical cell or tissue of a type which is susceptible to infection with human papillomavirus, e.g cervical epithelial cells. The cervical sample may be selected to include cell types/tissues which allow testing for expression of E6/E7 mRNA expression in the cervical mucosa and/or the upper layers of cervical epithelium. Suitable cervical samples include (but not exclusively) cervical swabs, cervical biopsies, cervical scrapings, samples removed with the use of brushes, lavage, scrapings, swabs and tampons etc., skin biopsies/warts, liquid-based cytology samples, also paraffin embedded tissues, and formalin or methanol fixed cells.

The assay for E6/E7 mRNA expression maybe carried out on a preparation of nucleic acid isolated from the cervical sample. The preparation of nucleic acid must include mRNA, however it need not be a preparation of purified poly A+ mRNA and preparations of total RNA or crude preparations of total nucleic acid containing both RNA and genomic DNA, or even crude cell lysates may also be used.

Detection of E6/E7 mRNA may be carried out using any suitable methodology, including mRNA amplification-based techniques such as for example RT-PCR, NASBA, TMA, rolling circle amplification etc. or techniques based on hybridisation.

In the methods of the invention, a treatment decision is made based on a positive result for expression of E6/E7 mRNA transcripts of at least one cancer-associated (high-risk) or carcinogenic HPV type. In this context “positive expression” of an mRNA is taken to mean expression above background. There is no absolute requirement for accurate quantitative determination of the level of E6/E7 mRNA expression, although in certain embodiments, the methods of the invention may comprise a quantitative determination of levels of mRNA expression. In order to provide a clear distinction between “positive expression” and “negative expression” a determination of “positive expression” may require the presence of more than 50 copies of the relevant mRNA per ml of sample tested or per total volume of sample tested (e.g. in the case of a liquid-based cytology sample).

The methods of the invention will preferably involve screening for E6/E7 mRNA using a technique which is able to detect specifically E6/E7 mRNA from one or more cancer-associated or carcinogenic HPV types, more preferably “high risk” cancer-associated HPV types.

In certain embodiments, subjects may be tested for expression of E6/E7 mRNA transcripts of at least one cancer-associated (high risk) or carcinogenic HPV type selected from the group consisting of HPV types 5, 6, 8, 11, 16, 18, 26, 31, 33, 35, 39, 45, 51, 52, 53, 56, 58, 59, 66, 68, 70, 73 and 82.

In one embodiment subjects may be tested for expression of E6/E7 mRNA transcripts of at least HPV type 16. In further embodiments, subjects may be tested for expression of E6/E7 mRNA transcripts of at least HPV types 16 and 18. In further embodiments, subjects may be tested for expression of E6/E7 mRNA transcripts HPV types 16, 18, and 45. In still further embodiments subjects may be tested for expression of E6/E7 mRNA transcripts of at least one HPV type selected from the group consisting of HPV types 16, 18, 31, 33 and 45. in still further embodiments subjects may be tested for expression of E6/E7 mRNA transcripts of at least HPV types 16, 18, 31, 33, 35, 45, 51, 52 and 58.

In a particularly preferred embodiment the “test and treat” methods may involve screening for E6/E7 mRNA using a technique which is able to detect E6 mRNA from HPV types 16, 18, 31 and 33, and preferably also 45. Most preferably, the method will specifically detect expression of E6/E7 mRNA from at least one of HPV types 16, 18, 31, 33, and preferably also 45, and most preferably all five types. However, women positive for positive for expression of E6/E7 from other types than 16, 18, 31, 33 and 45, e.g. 35, 39, 45, 52, 56, 58, 59, 66 and 68 may still be at risk of developing cervical carcinoma. Thus, the method may encompass screening for expression of E6/E7 mRNA from one or more of these HPV types, most preferably in addition to screening for E6/E7 mRNA from HPV types 16, 18, 31, 33 and 45. Certain HPV types exhibit a marked geographical or population distribution. Therefore, it may be appropriate to include primers specific for an HPV type known to be prevalent in the population/geographical area under test, for example in addition to screening for HPV types 16, 18, 31, 33 and 45. Screening for E6/E7 mRNA from HPV types 16, 18, 31, 33 and 45 can be conveniently carried out using the HPV Pretect Proofer kit, commercially available from Norchip A/S. Klokkarstua, Norway.

Unless otherwise stated, the terms “E6/E7 mRNA”, “E6/E7 transcripts” as used herein encompass all naturally occurring mRNA transcripts which contain all or part of the E6 open reading frame, including naturally occurring splice variants, and transcripts which contain all or part of the E7 open reading frame.

The methods of the invention may be based on specific detection of full length E6/E7 mRNA transcripts of some or all of the HPV types, which transcripts encode a full length E6 protein. In these embodiments presence of the full length E6/E7 mRNA is taken as a positive screening result.

The term “full length E6/E7 mRNA transcripts” excludes any of the naturally occurring splice variants, but encompasses bicistronic transcripts that encode functional full length E6 and E7 proteins. Four E6/E7 mRNA species have so far been described in cells infected with HPV 16, namely an unspliced E6 transcript and three spliced transcripts denoted E6*I, E6*II and E6*III (Smotkin D, et al., J Virol. 1989 March 63(3):1441 7; Smotkin D, Wettstein FO. Proc Natl Acad Sci USA. 1986 July 83(13):4680 4; Doorbar J. et al., Virology. 1990 September 178(1):254 62; Cornelissen M T, et al. J Gen Virol. 1990 May 71(Pt 5):1243 6; Johnson M A, et al. J Gen Virol. 1990 July 71(Pt 7):1473 9; Schneider Maunoury S, et al. J. Virol. 1987 October 61(10):3295 8; Sherman L, et al. Int J Cancer. 1992 February 50(3):356 64). All four transcripts are transcribed from a single promoter (p97) located just upstream of the second ATG of the E6 ORF. In the case of HPV 16, the term “full length E6/E7 transcripts” refers to transcripts which contain all or substantially all of the region from nucleotide (nt) 97 to nt 880 in the E6 ORF, inclusive of nt 97 and 880. Nucleotide positions are numbered according to standard HPV nomenclature (see Human Papillomavirus Compendium On Line, available via the internet or in paper form from HV Database, Mail Stop K710, Los Alamos National Laboratory, Los Alamos, N. Mex. 87545, USA).

In relation to HPV types other than HPV 16, “full length” E6/E7 transcripts may be taken to include transcripts which contain sequences homologous to the above-stated region of the HPV 16 E6/E7 transcript and to exclude E6 splice variants. Various sequence alignments of HPV types are publicly available via the Human Papillomavirus Compendium On Line.

Specific detection of full length E6/E7 mRNA transcripts (that is detection of full length E6/E7 transcripts in the absence of any spliced E6 transcripts) may be accomplished, for example, using primers or probes which are specific for the region which is present only in full length E6/E7 transcripts, not in splice variants.

The E6*I transcript exhibits loss of a coding sequence between nucleotides 226 and 409 (in HPV type 16) and the E*6II transcript exhibits loss of the coding sequence between nucleotides 226 and 526 (in HPV type 16). It is therefore preferred to use at least one primer or probe from the region located between nucleotides 226 and 409 of HPV type 16 or the homologous region from any other cancer-associated (high risk) or carcinogenic HPV type. Specificity for full length transcripts can be achieved by the use of a primer-pair in which one primer is specific for a sequence located within this region and the other primer is specific for a sequence located outside of this region or wherein both primers are specific for sequences within this region, preferably in conjunction with a probe specific for a sequence located within this region. In other embodiments it may be possible to use a primer-pair in which both primers are specific for sequences outside this region in combination with a probe specific for a sequence within the region in order to confer specificity for mRNA encoding full length E6.

Human female subjects whose cervical samples test positive for E6/E7 mRNA from at least one cancer associated (high risk) or carcinogenic HPV type are selected for direct treatment to reduce or eliminate transforming infection with human papillomavirus.

The methods of the invention may be used to “test and treat” any human female subject having potential exposure to infection with human papilloma virus. Current guidelines from the American College of Obstetricians and Gynecologists recommends routine screening in premenopausal women of 21 years and older and the test and treat methods may be applied in this screening population. The methods may be particularly useful in women of 30 years or older, or of 40 years and older.

The invention will be further understood with reference to the following experimental examples.

### EXAMPLES

### Material and methods

In the routine diagnostic practice at the University Hospital of North Norway (UNN), the E6/E7 mRNA test PreTect HPV-Proofer (Norchip A/S, Klokkarstua, Norway) is used in triage of ASC-US and LSIL for the detection of cervical dysplasia and cancer. The Department of Clinical Pathology receives cervical smears from the population of Troms and Finnmark County. About 23 000 cervical smears are analysed annually and between 2006 and 2009, smears from 65 041 women aged 25-69 years were analysed. A total of 3 206 women (4.9%) were diagnosed with ASC-US or LSIL. For these women, control cytology and the HPV E6/E7 mRNA test PreTect HPV-Proofer were recommended after 6 months. The compliance was high. Liquid based control cytology and mRNA results were received from 2 446 women (76.3% of the 3 206 women). Cells were extracted with the ThinPrep® 2000 (Cytyc Corporation, Marlborough, Mass., USA) for cytological examination. The HPV mRNA test uses a liquid based sample from the same material as cytology. The mRNA testing was performed according to the manufacturers instructions (NorChip AS, Klokkarstua, Norway) and in accordance with Norwegian national guidelines for HPV testing.

The cytological and histological diagnoses were collected from the diagnostic database (SymPathy) at the Department of Clinical Pathology, UNN. All biopsies with histological high-grade dysplasia and cancer (CIN2+) were evaluated by experienced pathologists. Biopsies with uncertain cellular changes were immunostained with p16 and eventually Ki67. Women with negative biopsies were followed with a new PAP-smear and mRNA test after 6 months.

### Results

Of the 2 446 women who received an mRNA test, 435 (18%) were positive. A total of 282 women (12%) had histological CIN2+ (moderate dysplasia, severe dysplasia or cancer). 65% of the 349 women with a positive mRNA test and that was biopsied had histological CIN2+ (PPV 0.65).

Of the 435 mRNA positive women 213 were positive for HPV type 16 (table 1). The PPV for histologically confirmed CIN2+ of the 176 women with mRNA from HPV type 16 and biopsy was 78%. For 48 women older than 40 years with E6/E7 mRNA from HPV 16, the PPV was 88% (table 1). For women with cytological HSIL and mRNA positive test (any of HPV types 16, 18, 31, 33 and 45), histological CIN2+ were confirmed in 96% of the cases.

### Discussion

Initially, a positive E6/E7 mRNA test followed by a negative biopsy was interpreted as a false positive mRNA result. However, when the woman was followed up with a new smear and a new mRNA test after 6 months, the mRNA test in most cases remained positive and the majority of these were confirmed to be histological CIN2+. This strongly indicates that a positive E6/E7 mRNA result represents the presence of cell abnormality/pre-cancer comparable to a morphological CIN2+ lesion. If CIN2+ is not detected by histology, the biopsy or the histological slide may not be representative for the underlying disease. Alternatively, a micro-lesion difficult to detect by colposcopy-directed biopsies and histology may be present.

It should be noted that E6/E7 mRNA expression does not correlate precisely with the “gold standard” of colposcopy-directed biopsy and histologically confirmed CIN2+/CIN3+ because colposcopy-directed biopsy and histology is a subjective method and the sensitivity of this technique is rather low.

The results presented here indicate that the correct consequence of a positive mRNA test for HPV type 16 in women 30 years or older should be direct treatment. In addition, women with cytological HSIL and a positive mRNA result for any of HPV types 16, 18, 31, 33 and 45 can be treated directly without the need for histological confirmation.

The results presented here suggest that the E6/E7 mRNA test (PreTect HPV-Proofer) may have no false positives, implying that positive women should be followed up by direct treatment.

These results support a “test and treat” clinical application of the PreTect HPV-Proofer test, i.e. direct treatment may be a feasible option for patients with a cytological ASCUS, LSIL or HSIL diagnosis having a positive result for E6/E7 mRNA, as measured for example by PreTect HPV-Proofer.

TABLE 1
HPV 16 mRNA positives

| | # positives | # positives with biopsies | PPV |
|---|---|---|---|
| All women | 213 | 176 | 78.4% |
| >30 years | 138 | 116 | 82.8% |
| >40 years | 57 | 48 | 87.5% |

1. A test and treat method of detecting and treating transforming infection with human papillomavirus (HPV) in a human female subject, the method consisting of:
testing a cervical sample from the subject for expression of E6/E7 mRNA transcripts from at least one cancer-associated (high risk) or carcinogenic HPV type;
making a treatment decision in a female human subject based on the outcome of the test for expression of E6/E7 mRNA transcripts of said at least one cancer-associated (high-risk) or carcinogenic HPV type, whereby a subject whose cervical sample tests positive for expression of E6/E7 mRNA transcripts from at least one cancer-associated (high risk) or carcinogenic HPV type is selected for direct treatment; and
treating said subject to reduce or eliminate transforming infection with human papillomavirus (HPV).

2. The method of claim 1 wherein the cervical sample is tested specifically for expression of full-length E6/E7 mRNA transcripts of at least one cancer-associated (high risk) or carcinogenic HPV type, which transcripts encode a full length E6 protein.

3. The method of claim 1 wherein the cervical sample is tested for expression of E6/E7 mRNA transcripts of at least one cancer-associated or carcinogenic HPV type selected from the group consisting of HPV types 5, 6, 8, 11, 16, 18, 26, 31, 33, 35, 39, 45, 51, 52, 53, 56, 58, 59, 66, 68, 70, 73 and 82.

4. The method of claim 1 wherein the cervical sample is tested for expression of E6/E7 mRNA transcripts of at least HPV type 16.

5. The method of claim 1 wherein the cervical sample is tested for expression of E6/E7 mRNA transcripts of at least HPV types 16 and 18.

6. The method of claim 1 wherein the cervical sample is tested for expression of E6/E7 mRNA transcripts of at least HPV types 16, 18, and 45.

7. The method of claim 1 wherein the cervical sample is tested for expression of E6/E7 mRNA transcripts of at least one HPV type selected from the group consisting of HPV types 16, 18, 31, 33 and 45.

8. The method of claim 1 wherein the cervical sample is tested for expression of E6/E7 mRNA transcripts of at least HPV types 16, 18, 31, 33, 35, 45, 51, 52 and 58.

9. A test and treat method of screening a population of female human subjects, selecting and treating individuals identified as having transforming infection with human papillomavirus (HPV) based on the outcome of a test for expression of E6/E7 mRNA transcripts of human papillomavirus (HPV), the method consisting of:
testing cervical samples from subjects in the population for expression of E6/E7 mRNA transcripts from at least one cancer-associated (high risk) or carcinogenic HPV type;
making a treatment decision based on the outcome of the test for expression of E6/E7 mRNA transcripts from at least one cancer-associated or carcinogenic HPV type in cervical samples from said subjects, whereby a subject whose cervical sample tests positive for expression E6/E7 mRNA transcripts from at least one cancer-associated or carcinogenic HPV type is selected for direct treatment; and
treating said subject to reduce or eliminate transforming infection with human papillomavirus (HPV).

10. The method of claim 9 wherein the cervical samples are tested specifically for expression of full-length E6/E7 mRNA transcripts of at least one cancer-associated or carcinogenic HPV type, which transcripts encode a full length E6 protein.

11. The method of claim 9 wherein the cervical samples are tested for expression of E6/E7 mRNA transcripts of at least one cancer-associated or carcinogenic HPV type selected from the group consisting of HPV types 5, 6, 8, 11, 16, 18, 26, 31, 33, 35, 39, 45, 51, 52, 53, 56, 58, 59, 66, 68, 70, 73 and 82.

12. The method of claim 9 wherein the cervical samples are tested for expression of E6/E7 mRNA transcripts of at least HPV type 16.

13. The method of claim 9 wherein the cervical samples are tested for expression of E6/E7 mRNA transcripts of at least HPV types 16 and 18.

14. The method of claim 9 wherein the cervical samples are tested for expression of E6/E7 mRNA transcripts of at least HPV types 16, 18, and 45.

15. The method of claim 9 wherein the cervical samples are tested for expression of E6/E7 mRNA transcripts of at least one HPV type selected from the group consisting of HPV types 16, 18, 31, 33 and 45.

16. The method of claim 9 wherein the cervical samples are tested for expression of E6/E7 mRNA transcripts of at least HPV types 16, 18, 31, 33, 35, 45, 51, 52 and 58.

17. A test and treat method of triaging female subjects having a previous diagnosis of ASC-US, low-grade cervical lesions (LSIL) or high-grade cervical lesions (HSIL) by cytology, selecting and treating individuals identified as having transforming infection with human papillomavirus (HPV) based on the outcome of a test for expression of E6/E7 mRNA transcripts of human papillomavirus (HPV), the method consisting of:
testing a cervical sample from a subject having a previous diagnosis of ASC-US, low-grade cervical lesions (LSIL) or high-grade cervical lesions (HSIL) by cytology for expression of E6/E7 mRNA transcripts from at least one cancer-associated (high risk) or carcinogenic HPV type;
making a treatment decision based on the outcome of the test for expression of E6/E7 mRNA transcripts from at least one cancer-associated (high risk) or carcinogenic HPV type in said cervical sample, whereby a subject whose cervical sample test positive for expression of E6/E7 mRNA transcripts from at least one cancer-associated or carcinogenic HPV type is selected for direct treatment; and
treating said subject to reduce or eliminate transforming infection with human papillomavirus (HPV).

18. The method of claim 17 wherein the cervical sample is tested specifically for expression of full-length E6/E7 mRNA transcripts of at least one cancer-associated (high risk) or carcinogenic HPV type, which transcripts encode a full length E6 protein.

19. The method of claim 17 wherein the cervical sample is tested for expression of E6/E7 mRNA transcripts of at least one HPV type selected from the group consisting of HPV types 5, 6, 8, 11, 16, 18, 26, 31, 33, 35, 39, 45, 51, 52, 53, 56, 58, 59, 66, 68, 70, 73 and 82.

20. The method of claim 17 wherein the cervical sample is tested for expression of E6/E7 mRNA transcripts of at least HPV type 16.

21. The method of claim 17 wherein the cervical sample is tested for expression of E6/E7 mRNA transcripts of at least HPV types 16 and 18.

22. The method of claim 17 wherein the cervical sample is tested for expression of E6/E7 mRNA transcripts of at least HPV types 16, 18, and 45.

23. The method of claim 17 wherein the cervical sample is tested for expression of E6/E7 mRNA transcripts of at least one HPV type selected from the group consisting of HPV types 16, 18, 31, 33 and 45.

24. The method of claim 17 wherein the cervical sample is tested for expression of E6/E7 mRNA transcripts of at least HPV types 16, 18, 31, 33, 35, 45, 51, 52 and 58.

25. A test and treat method of triaging female subjects who previously tested positive for the presence of DNA from at least one cancer-associated (high risk) or carcinogenic HPV type, selecting and treating individuals identified as having transforming infection with human papillomavirus (HPV) based on the outcome of a test for expression of E6/E7 mRNA transcripts of human papillomavirus (HPV), the method consisting of:
testing a cervical sample from a subject who previously tested positive for the presence of DNA from at least one cancer-associated (high risk) or carcinogenic HPV type for expression of E6/E7 mRNA transcripts from at least one cancer-associated (high risk) or carcinogenic HPV type;
making a treatment decision based on the outcome of the test for expression of E6/E7 mRNA transcripts from at least one cancer-associated or carcinogenic HPV type in said cervical sample, whereby a subject whose cervical sample tests positive for expression E6/E7 mRNA transcripts from at least one cancer-associated or carcinogenic HPV type is selected for direct treatment; and
treating said subject to reduce or eliminate transforming infection with human papillomavirus (HPV).

26. The method of claim 25 wherein the cervical sample is tested specifically for expression of full-length E6/E7 mRNA transcripts of at least one cancer-associated or carcinogenic HPV type, which transcripts encode a full length E6 protein.

27. The method of claim 25 wherein the cervical sample is tested for expression of E6/E7 mRNA transcripts of at least one cancer-associated or carcinogenic HPV type selected from the group consisting of HPV types 5, 6, 8, 11, 16, 18, 26, 31, 33, 35, 39, 45, 51, 52, 53, 56, 58, 59, 66, 68, 70, 73 and 82.

28. The method of claim 25 wherein the cervical sample is tested for expression of E6/E7 mRNA transcripts of at least HPV type 16.

29. The method of claim 25 wherein the cervical sample is tested for expression of E6/E7 mRNA transcripts of at least HPV types 16 and 18.

30. The method of claim 25 wherein the cervical sample is tested for expression of E6/E7 mRNA transcripts of at least HPV types 16, 18, and 45.

31. The method of claim 25 wherein the cervical sample is tested for expression of E6/E7 mRNA transcripts of at least one HPV type selected from the group consisting of HPV types 16, 18, 31, 33 and 45.

32. The method of claim 25 wherein the cervical sample is tested for expression of E6/E7 mRNA transcripts of at least HPV types 16, 18, 31, 33, 35, 45, 51, 52 and 58.
